(19) United States
(12) Patent Application Publication
(10) Pub. No.: US 2026/0123832 A1
(43) Pub. Date: May 7, 2026

(54) SYSTEM FOR A FULL-FIELD OCT EYE MEASUREMENT

(71) Applicant: INCELLVU S.A., Warszawa (PL)

(72) Inventor: Maciej Daniel WOJTKOWSKI, Torun (PL)

(73) Assignee: INCELLVU S.A., Warszawa (PL)

(21) Appl. No.: 19/117,821

(22) PCT Filed: Oct. 2, 2023
(86) PCT No.: PCT/EP2023/077276

§ 371 Date: Apr. 2, 2025

(30) Foreign Application Priority Data
Oct. 5, 2022 (EP) 22461614.4

(51) Int. Cl. A61B 3/10; A61B 3/14

(52) U.S. Cl. CPC A61B 3/102; A61B 3/14

(57) ABSTRACT

A system for a full-field OCT eye measurement, comprising:—a source (1) of a light beam (2),—a detector (3), preferably in form of a camera,—an interferometer, comprising a reference arm (4) and a sample arm (5),—a beam splitter (6), configured to receive the light beam (2) and to split the light beam (2) into a sample beam (7) and a reference beam (8) and further configured to direct the sample beam (2) to the sample arm (5) and the reference beam (8) to the reference arm (4) respectively, wherein the sample arm (5) is configured for guiding the sample beam (7) through a point on the cornea of a subject's eye (9) into the subject's eye (9) and back to the detector through the beam splitter (6), wherein the reference arm (4) is configured for guiding the reference beam (8) to a mirror (10) and back to the detector (3) through the beam splitter (6), wherein said detector (3) is configured and programmed for comparing the sample beam (7) and the reference beam (8) for detecting diseases of the subject's eye (9). The system is characterized in that it comprises an arrangement for decreasing the intensity of the sample beam (7) passing through the point of the cornea of the subject's eye (9).

### TECHNICAL FIELD

The present invention relates to full-field OCT (optical coherence tomography) eye measurement, in particular to system for a full-field OCT eye measurement, especially with laser light, which raises safety concerns, specifically in the imaging techniques from the full-field family.

### BACKGROUND

In the full-field modalities the eye illuminating beam is being focused in front of the patient's eye so it can be naturally collimated by the patient's eye lens to uniformly cover the retina. The natural focal length of the human eye lens is close to the eyeball internal length which equals approximately 20 mm. The eye lens focuses the incoming collimated beam onto the retina. However, in the full-field retina imaging methods the situation is inverted and it is desired to obtain a uniform illumination of the retina by collimating the beam on it. To achieve this, a focus is formed in front of the eye in the focal distance from the lens which is similar to that between the lens and the retina.

Such proximity of the beam with respect to the front of the eye poses a threat of exceeding the laser safety norms for the light intensity allowed on the cornea and causing consequent damage. This danger may become unnoticed by the operators and the system designers due to the fact of the retina being the imaging object while the cornea and the lens are only used as transmissive optics. Especially during the initial instrument alignment when the focus position gets shifted in the search of optimal image quality, the focus is likely to approach the cornea to the distance too close for the power density to be still compliant with the safety norms.

Document U.S. Pat. No. 10,758,123B2 discloses an ophthalmological microscope system, having an illumination system that projects illumination light onto a subject's eye. A light receiving system guides returning light of the illumination light to an image sensor or an eyepiece system. An interference optical system splits light into measurement light and reference light and detects interference light generated from returning light of the measurement light and the reference light. A designation unit is used for designating an operation mode. When an observation priority mode (or an OCT priority mode) has been designated, a controller executes first light amount control that restricts light amount of the measurement light (or second light amount control that restricts light amount of the illumination light) to make total light amount of the illumination light and the measurement light equal to or less than a predetermined value. This solution says nothing about the position of the light beam on the eye. In particular, it is silent about any way of protecting the eye.

Document U.S. Pat. No. 7,061,622B2 describes an optical coherence tomography (OCT) system including an interferometer that provides illuminating light along a first optical path to a sample and an optical delay line and collects light from the sample along a second optical path remitted at several scattering angles to a detector. In one embodiment, illuminating light is directed along a number of incident light paths through a focusing lens to a sample. The light paths and focusing lens are related to the sample and to both the incident light source and the detector. In another embodiment, a focusing system directs light to a location in the sample. A transmission grating or acousto-optic modulator directs light from the sample at an angle representative of the wavelength of the incident light on the transmission grating or acousto-optic modulator. This solution does not solve the problem of too high intensity of the light beam falling on a fixed point on the cornea.

Document CN114903425A discloses the invention that provides a visible light OCT (Optical Coherence Tomography) device and method for reducing eye watching fatigue during focusing. A first light source is connected with an optical coupler through an optical fiber, the optical coupler is connected with the input end of a first collimator through an optical fiber, the optical coupler is connected with a spectrograph through an optical fiber, and the spectrograph is communicated with a processing terminal. The first scanning galvanometer is used for scanning output light of the first collimator in a first direction, and the second scanning galvanometer is used for scanning reflected light of the first scanning galvanometer in a second direction to obtain first reflected light; the second light source is connected with the input end of the second collimator through an optical fiber, the third scanning galvanometer is used for scanning output light of the second collimator in the first direction, and the second scanning galvanometer is used for scanning reflected light of the third scanning galvanometer in the second direction to obtain second reflected light; the first focusing lens and the second focusing lens form a 4f system, and the 4f system is used for receiving the first reflected light and the second reflected light and outputting light acting on human eyes; according to the invention, visual fatigue is reduced. The method of solving the problem proposed in this solution also brings with it the problem of delivering too intense light beam at one point in the eye.

None of the documents in the prior art discloses a solution in which light intensity that is delivered to the cornea is decreased and solves the problem of increasing safety of the examined eye.

### BRIEF DESCRIPTION OF THE INVENTION

The essence of the invention is a system for a full-field OCT eye measurement, comprising:
a source of a light beam,
a detector, preferably in form of a camera,
an interferometer, comprising a reference arm and a sample arm,
a beam splitter, configured to receive the light beam and to split the light beam into a sample beam and a reference beam and further configured to direct the sample beam to the sample arm and the reference beam to the reference arm respectively,
   wherein the sample arm is configured for guiding the sample beam through a point on the cornea of a subject's eye into the subject's eye and back to the detector through the beam splitter,
   wherein the reference arm is configured for guiding the reference beam to a mirror and back to the detector through the beam splitter,
   wherein said detector is configured and programmed for comparing the sample beam and the reference beam for detecting diseases of the subject's eye.

The system is characterized in that it comprises an arrangement for decreasing the intensity of the sample beam passing through the point of the cornea of the subject's eye.

Preferably, the arrangement comprises means for moving the sample beam parallel to its original direction so as to change the point of the cornea through which the sample beam enters the subject's eye.

Preferably, said means for moving are configured for moving the source of the light beam.

Preferably, said means for moving comprises a movable lens that is placed between the beam splitter and the subject's eye.

Preferably, the arrangement comprises means for dithering the sample beam.

Preferably, said means for dithering are configured for temporarily switching on and off the source of the light.

Preferably, said means for dithering comprises a movable diaphragm or movable mirror placed between the beam splitter and the subject's eye or between the source of the light beam and the beam splitter.

Preferably, the arrangement comprises means for splitting the sample beam into at least two separate sample sub-beams and for directing the at least two sample sub-beams into the subject's eye through at least two different points on the cornea of the subject's eye, wherein said means for splitting are placed between the beam splitter and the subject's eye.

Preferably, that it comprises limiting means configured for limiting the position of the focus of the sample beam with respect to the cornea of the subject's eye such that the distance between the focus of the sample beam and the cornea of the subject's eye is not smaller than a predefined minimal distance.

Preferably, the limiting means comprises a camera.

### BRIEF DESCRIPTION OF THE DRAWINGS

Preferred embodiment of the present invention are presented in a more detailed way with reference to the attached drawing, in which:

FIG. 1 is a scheme of a system for a full-field OCT eye measurement,

FIG. 2 is a scheme of the system according to the first embodiment,

FIG. 3 is a scheme of the system according to the second embodiment,

FIG. 4 is a scheme of the system according to the third embodiment,

FIG. 5 is a scheme of the system according to the fourth embodiment.

FIG. 6 is a scheme of the system according to the fifth embodiment.

### DETAILED DESCRIPTION

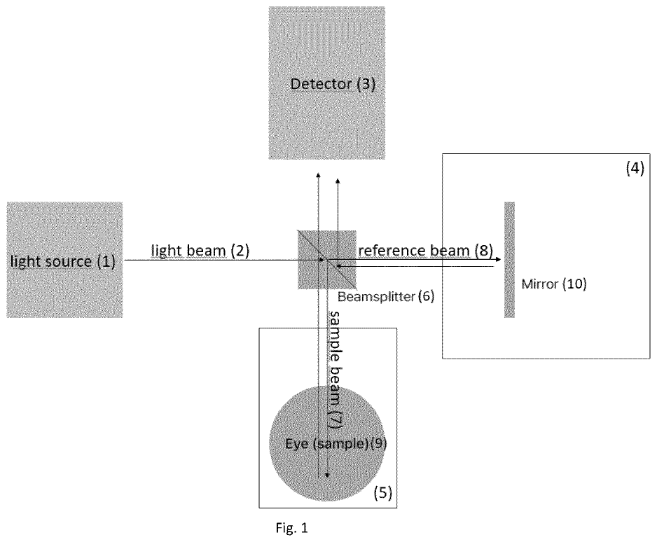

A system for a full-field OCT eye measurement is schematically shown in FIG. 1. The system comprises a source 1 of the light beam 2, a detector 3, that is preferably a 2D detector, preferably in form of a camera, an interferometer, comprising a reference arm 4 and a sample arm 5. The system comprises also a beam splitter 6 that is configured to receive the light beam 2 and to split the light beam 2 into a sample beam 7 and a reference beam 8 and further configured to direct the sample beam 2 to the sample arm 5 and the reference beam 8 to the reference arm 4 respectively.

The sample arm 5 of the interferometer is configured for guiding the sample beam 7 through a point on the cornea of a subject's eye 9 into the subject's eye 9 and back to the detector through the beam splitter 6.

The reference arm 4 of the interferometer is configured for guiding the reference beam 8 to a mirror 10 and back to the detector 3 through the beam splitter 6.

What is more, the detector 3 is configured and programmed for comparing the sample beam 7 and the reference beam 8 for detecting diseases of the subject's eye 9.

The system comprises an arrangement for decreasing the intensity of the sample beam 7 passing through the point of the cornea of the subject's eye 9.

The source 1 of the light beam 2 may be tunable. It comprises a light delivery system that can deliver light beam 2 through a fiber or free space optics or both. Each or any of the light paths of the: light beam 2, sample beam 7, reference beam 8 or sub-beam(s) can contain optics to shape said beams and/or collect signal, which can be in form of lenses or mirrors.

Respectively, reference arm 4 and/or sample arm 5 may be capable of receiving and delivering any of the beams through fiber, free space optics or both.

What is more, such configuration of the system according to the invention allows to focus the sample beam 7 in front of the subject's eye 9 to get parallel beams on retina after passing the eye lens. Focus of the sample beam 7 forms a hazardous point.

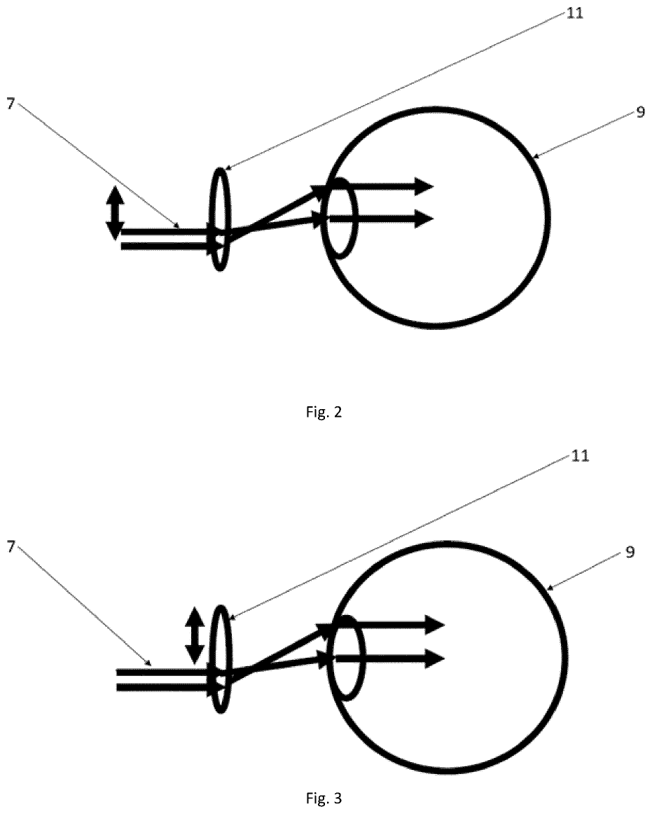

According to the first embodiment of the invention, showed in details in FIG. 2, the arrangement comprises means for moving the sample beam 7 parallel to its original direction so as to change the point of the cornea through which the sample beam 7 enters the subject's eye 9. Said means for moving are configured for moving the source 1 of the light beam 2.

In this embodiment, the incoming light beam 2 can be shifted sideways parallel to the its original direction by deviating it. It can be overcome with any standard means such as a galvo scanner, an acousto-optic deflector or electro-optic deflector. The movement can be periodic or, according to any other temporal scheme, deterministic or random. Moving the light beam 2 around helps avoiding stationary illumination through a fixed spot which decreases the integrated heat absorbed by the point on the cornea of the subject's eye 9 in the front of the subject's eye 9 and thus relaxes the safety concerns. The single movement may bring the light beam 2 to a new point on the cornea of a subject's eye 9 completely or only partially, leaving the illumination active in the previously used point on the cornea of the subject's eye 9. The shift of the light beam 2 can be continuous or performed as a switching act (“jump”) to a new place.

FIG. 3 shows the second embodiment of the invention, in which means for moving the light beam 2 comprises a movable lens 11 that is placed between the beam splitter 6 and the subject's eye 9.

In this embodiment the same result is obtained as in the previous one but the means to it is a transmissive optical element, preferably a lens 11, through which the light beam 2 is directed onto the eye, which gives the same result, i.e. displacement of the beam entry point to the eye to avoid stationary beam on the cornea. The movement is in form of a shift or a dither of the movable lens 11 and this movement can be continuous or jumping, periodic, aperiodic and deterministic or random.

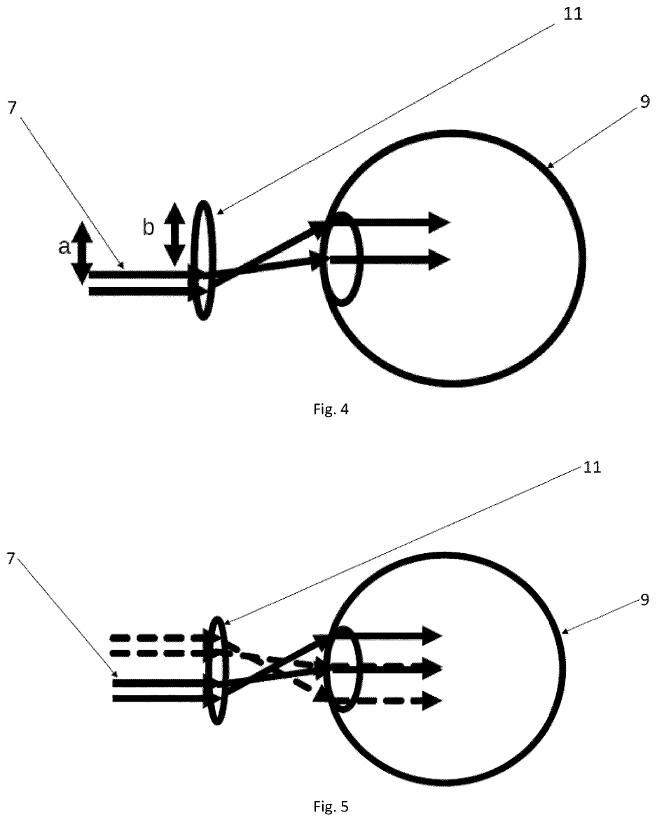

The third embodiment is shown in FIG. 4. It discloses the arrangement which comprises means for moving (a) the sample beam 7 parallel to its original direction so as to change the point of the cornea through which the sample beam 7 enters the subject's eye 9 and the movable lens 11 that is placed between the beam splitter 6 and the subject's eye 9, wherein the movement (b) is in form of a shift or a dither of the movable lens 11 and this movement can be continuous or jumping, periodic, aperiodic and deterministic or random.

The system according to the invention may comprise means for dithering the sample beam 7. Said means for dithering may be implemented together with or independent from any other means as described above or below or it can be the only means for decreasing the intensity of the sample beam 7 passing through the point of the cornea of the subject's eye 9, reaching this effect by dithering the sample beam 7.

Preferably, the means for dithering are configured for temporarily switching on and off the source 1 of the light. In this arrangement, the light beam 2 remains in the same place but is being temporarily switched off and on or generally modulated to obtain the same result, i.e. thermal relief for the cornea tissue. Dumping of the light beam 2 can be realized with a beam block (periodic, e.g. rotating chopper or on demand, e.g. with a shutter) or with beam deflection far enough to avoid hitting the subject's eye 9 altogether (e.g. with an acousto-or electro-optic modulator). This light beam 2 modulation may be complete (i.e. with no light on the eye when modulation engaged) or partial, i.e. with the light beam 2 intensity only decreased compared to the working value.

The fourth aspect of the invention is shown in FIG. 5. In this embodiment the arrangement comprises means for splitting the sample beam 7 into at least two separate sample sub-beams and for directing the at least two sample sub-beams into the subject's eye 9 through at least two different points on the cornea of the subject's eye 9. The means for splitting are placed between the beam splitter 6 and the subject's eye 9. This results in decreasing the light beam 2 intensity experienced by the front of the tissue of the subject's eye 9.

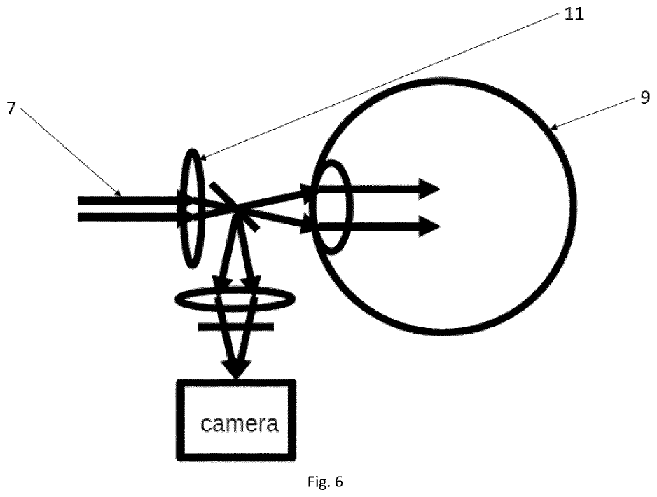

The system, according to fifth embodiment is shown in FIG. 6. It comprises limiting means configured for limiting the position of the focus of the sample beam 7 with respect to the cornea of the subject's eye 9 such that the distance between the focus of the sample beam 7 and the cornea of the subject's eye 9 is not smaller than a predefined minimal distance. Preferably, the distance from the cornea of the subject's eye 9 is in range between 1 and 2 cm.

Preferably, the limiting means comprises a camera. If the intensity of the light beam 2 on the camera exceeds a predefined value as a result of a change in focusing conditions, the sample beam 7 is blocked or deflected from the eye to avoid too high optical power density on the cornea of the subject's eye 9. Alternatively, when the power density (intensity of light beam 2) on the camera is too high, another means to decrease it is decreasing the total optical power of the light beam 2 without blocking the light beam 2.

1. A system for a full-field OCT eye measurement, comprising:
a source of a light beam,
a detector, preferably in form of a camera,
an interferometer, comprising a reference arm and a sample arm,
a beam splitter, configured to receive the light beam and to split the light beam into a sample beam and a reference beam and further configured to direct the sample beam to the sample arm and the reference beam to the reference arm respectively,
wherein the sample arm is configured for guiding the sample beam through a point on the cornea of a subject's eye into the subject's eye and back to the detector through the beam splitter,
wherein the reference arm is configured for guiding the reference beam to a mirror and back to the detector through the beam splitter,
wherein said detector is configured and programmed for comparing the sample beam and the reference beam for detecting diseases of the subject's eye,
characterised in that it comprises an arrangement for decreasing the intensity of the sample beam passing through the point of the cornea of the subject's eye, wherein the sample beam is configured to be focused in front of the subject's eye,
wherein the arrangement comprises means for moving the sample beam parallel to its original direction so as to change the point of the cornea through which the sample beam enters the subject's eye, and wherein said means for moving are configured for moving the source of the light beam.

2. (canceled)

3. (canceled)

4. The system according to claim 1, characterised in that said means for moving comprises a movable lens that is placed between the beam splitter and the subject's eye.

5. The system according to claim 1, characterised in that the arrangement comprises means for dithering the sample beam.

6. The system according to claim 5, characterised in that said means for dithering are configured for temporarily switching on and off the source of the light.

7. The system according to claim 5, characterised in that said means for dithering comprises a movable diaphragm or movable mirror placed between the beam splitter and the subject's eye or between the source of the light beam and the beam splitter.

8. The system according to claim 1, characterised in that the arrangement comprises means for splitting the sample beam into at least two separate sample sub-beams and for directing the at least two sample sub-beams into the subject's eye through at least two different points on the cornea of the subject's eye, wherein said means for splitting are placed between the beam splitter and the subject's eye.

9. The system according to claim 1, characterised in that it comprises limiting means configured for limiting the position of the focus of the sample beam with respect to the cornea of the subject's eye such that the distance between the focus of the sample beam and the cornea of the subject's eye is not smaller than a predefined minimal distance.

10. The system according to claim 9, characterised in that the limiting means comprises a camera.
